(19) United States
(12) Patent Application Publication
(10) Pub. No.: US 2026/0026478 A1
(43) Pub. Date: Jan. 29, 2026

(54) CONSTRUCTION METHOD AND USE OF Thy1-SNCA; Clu GENE KNOCKOUT MOUSE MODEL

(71) Applicant: Jianghan University, Wuhan (CN)

(72) Inventors: Yiyuan XIA, Wuhan (CN); LAN ZHANG, Wuhan (CN); Ji WANG, Wuhan (CN); Xiji SHU, Wuhan (CN)

(73) Assignee: Jianghan University, Wuhan (CN)

(21) Appl. No.: 19/180,042

(22) Filed: Apr. 15, 2025

(30) Foreign Application Priority Data
Aug. 8, 2024 (CN) 202411086460.0

(51) Int. Cl. A01K 67/0276; C12N 9/22; C12N 15/11

(52) U.S. Cl. CPC A01K 67/0276; C12N 9/226; C12N 15/111; A01K 2217/075; A01K 2217/15; A01K 2227/105; A01K 2267/0318

(57) ABSTRACT

A construction method and use of a Thy1-SNCA;Clu gene knockout mouse model are provided. The construction method includes: subjecting a Thy1-SNCA mouse to crossing with a Clu-KO mouse to obtain an F1 generation, and then conducting identification to obtain a Thy1-SNCA;Clu−/+ mouse; and subjecting the Thy1-SNCA;Clu−/+ mouse to backcrossing with the Clu-KO mouse to obtain an F2 generation, and then conducting identification to obtain a mouse with a target genotype, recorded as a Thy1-SNCA;Clu−/− mouse. The Thy1-SNCA;Clu−/− mouse can effectively shorten a time point for the onset of Parkinson's disease (PD)-like movement disorders, thereby shortening an experimental period. The mouse can provide more model mouse options for PD research.

### CROSS-REFERENCE TO RELATED APPLICATIONS

This application claims priority to Chinese Patent Application No. 202411086460.0 with a filing date of Aug. 8, 2024. The content of the aforementioned application, including any intervening amendments thereto, is incorporated herein by reference.

### CROSS-REFERENCE TO A SEQUENCE LISTING

This application includes a Sequence Listing submitted electronically as a text file named 8401SEQ-list.xml, created on Mar. 12, 2023, with a size of 13,428 bytes. The Sequence Listing is incorporated by reference herein.

### TECHNICAL FIELD

The present disclosure relates to the technical field of Parkinson's disease (PD) research, and in particular to a construction method and use of a Thy1-SNCA;Clu gene knockout mouse model.

### BACKGROUND

Parkinson's disease (PD) is the second most common neurodegenerative disease, characterized by motor and non-motor disorders. Pathological features of the PD include the loss of a large number of dopaminergic neurons in the substantia nigra and the formation of Lewy bodies (LBs). α-Synuclein (α-syn, with a gene called SNCA) is a main component of the LBs.

Currently, animal models of PD include drug-induced neurotoxin models (such as MPTP, 6-OHDA, and rotenone models) and transgenic models (such as SNCA, Parkin, LRRK2, PINK1, and DJ-1 models). The MPTP model shows simple operations and selective damage to dopamine neurons, and can successfully replicate the characteristics of PD and be used in movement disorder research. However, the MPTP model can only simulate movement disorders and can't simulate other non-motor symptoms of the PD. The 6-OHDA model can selectively destroy dopamine neurons with a high destruction effect on the dopamine neurons, and is used in movement disorder research and drug screening. However, this model can non-specifically destroy dopamine neurons and has a low survival rate. The rotenone model can simulate mitochondrial dysfunction and oxidative stress, and is therefore used in mitochondrial function research. However, such a model has complex operations and low survival rate. Modifications in SNCA transgenic models include mutations (A53T, A30P, and E46K) or duplications. The SNCA models are helpful in studying α-nucleoprotein-related degeneration and the relationship between genetic and environmental factors in PD. However, some of these models do not show significant dopaminergic neuron loss or show cell apoptosis patterns that are inconsistent with human PD pathology. LRRK2 in the LRRK2 transgenic models is a pathogenic gene of familial PD, showing a modification mode of mutation (G2019S and R1441C/G). Such models can be used for LRRK2-targeted drug testing and research on LRRK2 function, but shows motor defects without obvious dopaminergic neuron loss and lacks LB pathological manifestations. Mutation or knockout of ubiquitin ligase in the Parkin transgenic models may lead to the accumulation of neurotoxic substrates. This model is useful for studying Parkin function, but phenotypic data are currently lacking. The DJ-I gene knockout model can be effectively combined with a neurotoxin model. Although there are motor defects, this model demonstrates lack of LB pathology, no significant dopamine neuron loss, and little phenotypic data. In PINK-1 transgenic models, PINK-1 is a neuroprotective kinase with modification methods including mutation and knockout. For example, G309D-PINK1 mice can show dopamine reduction and motor defect phenotypes. However, most PINK-1 models do not show a decrease in dopaminergic neurons or dopamine levels. The above indicates that the existing PD models cannot fully reflect the motor and non-motor pathological characteristics of PD due to certain defects to varying degrees.

Thy1-SNCA, one of the most widely used animal models of PD, overexpresses human α-syn under the Thy1 promoter. The overexpression begins on day 10 after birth, and then increases steadily after the day 10. However, there are certain differences in the overexpression between different brain regions. An expression level of α-syn in the hippocampus continues to increase from 2 to 6 months of age. The expression of α-syn in the striatum decreases slightly or remains unchanged from 2 to 6 months of age, and then decreases by approximately 40% at 14 months of age. An expression level of tyrosine hydroxylase (TH) also decreases at 14 months of age, by only approximately 20%. More importantly, mortality and morbidity as well as general health of Thy1-SNCA mice before 14 months of age are similar to those of wild-type mice. PD-like movement disorders are relatively obvious only around 14 months of age.

### SUMMARY OF PRESENT INVENTION

In view of the shortcomings of PD animal models in the prior art, an objective of the present disclosure is to develop a novel PD animal model with a short experimental cycle and stable pathological changes.

In order to achieve the above objective, the present disclosure adopts the following technical solutions:

In a first aspect, the present disclosure provides a construction method of a Thy1-SNCA;Clu gene knockout mouse model, including the following steps:
subjecting a Thy1-SNCA mouse to crossing with a Clu-KO mouse to obtain an F1 generation, and then conducting identification to obtain a Thy1-SNCA;Clu−/+ mouse; and
subjecting the Thy1-SNCA;Clu−/+ mouse to backcrossing with the Clu-KO mouse to obtain an F2 generation, and then conducting identification to obtain a mouse with a target genotype, recorded as a Thy1-SNCA;Clu−/− mouse.

In the construction method of the present disclosure, a Clu gene of the Clu-KO mouse exhibits functional loss relative to that of the wild-type mouse.

The inventors have found that Clu can mediate the degradation of α-syn to alleviate PD symptoms; and complete knockout of the Clu in mice can increase the aggregation of α-syn in vivo. Based on the above finding, the inventors speculate that knocking out Clu is a key step in accelerating the occurrence of PD-like symptoms of the SNCA gene, and then establish the Thy1-SNCA;Clu gene knockout mouse model. Experimental data show that the Thy1-SNCA;Clu gene knockout mouse model shows a large amount of tyrosine hydroxylase loss, a large amount of aggregated phosphorylated α-syn, and significant asymmetry of gait at 6 months of age. These phenomena occur much earlier than those in the 14-month-old Thy1-SNCA mice, thus effectively shortening an experimental period.

Preferably, the Clu-KO mouse lacks a sequence shown in SEQ ID NO: 1 relative to a wild-type mouse. The Clu gene is located on chromosome 14 and has five transcripts. In some examples of the present disclosure, the Clu-KO mouse is obtained by knocking out the E3 to E4 regions of exon (where a sequence is specifically shown in SEQ ID NO: 1, with a total length of 2,636 bp). The Thy1-SNCA;Clu gene knockout mouse model obtained by crossing the Clu-KO mouse with the Thy1-SNCA mouse and then backcrossing is superior to a traditional animal model in terms of stability, pathology, behavioral characteristics, and time of occurrence, and shows a high animal survival rate.

Preferably, the Clu-KO mouse is constructed using a CRISPR/Cas9 system. In some examples of the present disclosure, a construction process of the Clu-KO mouse includes the following steps: microinjecting the CRISPR/Cas9 system and a homologous recombination vector (Donor vector) into a fertilized egg of a female C57BL/6N background mouse, transplanting the fertilized egg into a pseudo-pregnant female mouse to obtain a recipient mouse, allowing the recipient mouse to become pregnant and give birth to a newborn mouse of a F0 generation, and then extracting a genomic DNA of the newborn mouse to allow identification to obtain the Clu-KO mouse.

More preferably, gRNA target sequences in the CRISPR/Cas9 system are shown in SEQ ID NO: 2 and SEQ ID NO: 3.

More preferably, when the Clu-KO mouse lacks the sequence shown in SEQ ID NO: 1, the Clu-KO mouse can be identified by a primer pair shown in SEQ ID NO: 4 and SEQ ID NO: 5. Moreover, an individual is the Clu-KO mouse when a product size is 514 bp, and is the wild-type mouse when the product size is 3,150 bp.

Preferably, a primer pair for identifying the Thy1-SNCA mouse or Thy1-SNCA;Clu−/+ mouse includes: a primer pair having sequences shown in SEQ ID NO: 7 to SEQ ID NO: 8 and a primer pair having sequences shown in SEQ ID NO: 9 to SEQ ID NO: 10.

Preferably, a primer pair for identifying the mouse with the target genotype includes: a primer pair having sequences shown in SEQ ID NO: 7 to SEQ ID NO: 8, a primer pair having sequences shown in SEQ ID NO: 9 to SEQ ID NO: 10, a universal primer having a sequence shown in SEQ ID NO: 4, and a reverse primer having a sequence shown in SEQ ID NO: 5 or SEQ ID NO: 6. Specifically, an extracted genomic DNA is subjected to PCR using the primer pair, and a mouse individual having both Thy1-SNCA and Clu−/− genotypes is retained, thereby obtaining the mouse with the target genotype.

In a second aspect, the present disclosure provides use of a Thy1-SNCA;Clu gene knockout mouse model constructed by the construction method in preparation of a model for PD. Pathological and behavioral tests of the model reveal that the model shows a large amount of tyrosine hydroxylase loss, a large amount of aggregated phosphorylated α-syn, and significant asymmetry of gait at 6 months of age. Therefore, the model has more advantages than the Thy1-SNCA model, and can be a desirable substitute for Thy1-SNCA mice in PD research.

In a third aspect, the present disclosure provides use of a Thy1-SNCA;Clu gene knockout mouse model constructed by the construction method in study on a function and a mechanism of action of an SNCA gene.

In a fourth aspect, the present disclosure provides use of the Clu-KO mouse in exploring function, mechanism of action, and molecular pathways involved in the Clu gene in nervous system diseases, as well as use of the Clu-KO mouse as an animal model for drug screening using Clu as a molecular target.

Compared with the prior art, the present disclosure has the following beneficial effects:

The inventors discover for the first time that Clu can mediate the degradation of α-syn to alleviate PD symptoms; based on this theoretical breakthrough, a novel PD animal model, namely the Thy1-SNCA;Clu−/− mouse, is developed in the present disclosure. Compared with the existing Thy1-SNCA mice, the model can effectively shorten a time point for the onset of PD-like movement disorders, thereby shortening an experimental period.

In the present disclosure, the Clu-KO mouse is preferably obtained by knocking out E3 to E4 regions of exon relative to the wild-type mouse. The Thy1-SNCA;Clu gene knockout mouse model obtained by crossing the Clu-KO mouse with the Thy1-SNCA mouse and then backcrossing is superior to a traditional animal model in terms of stability, pathology, behavioral characteristics, and time of occurrence, and shows a high animal survival rate.

In the present disclosure, the construction method is simple and easy and the model has a high survival rate. The model can not only be used as a PD model, but also can be used to explore the role of SNCA and Clu in nervous system diseases, related mechanisms, and possible molecular pathways involved. The present disclosure further provides a novel animal model for drug screening with Clu as a molecular target.

### BRIEF DESCRIPTION OF THE DRAWINGS

FIG. 1 shows a schematic diagram of a strategy for the Clu-KO gene knockout mouse in Example 1 of the present disclosure;

FIG. 2 shows the detection of PCR products of the Clu-KO gene knockout mouse in Example 1 of the present disclosure;

FIG. 3 shows the sequencing results of the PCR products of the Clu-KO gene knockout mouse in Example 1 of the present disclosure;

FIG. 4 shows an electrophoretic diagram of the Thy1-SNCA genotype identification of the F1 generation mouse in Example 2 of the present disclosure;

FIG. 5 shows an electrophoretic diagram of the Thy1-SNCA genotype identification of the F2 generation mouse in Example 2 of the present disclosure;

FIG. 6 shows an electrophoretic diagram of the Clu-KO genotype identification of the F2 generation mouse in Example 2 of the present disclosure;

FIG. 7 shows the ThS staining results of the striatum and substantia nigra compacta (SNc) of different mouse models at 1 month of age in Example 3 of the present disclosure;

FIG. 8 shows the ThS staining results of the striatum and substantia nigra compacta (SNc) of different mouse models at 3 months of age in Example 3 of the present disclosure;

FIG. 9 shows the ThS staining results of the striatum and substantia nigra compacta (SNc) of different mouse models at 6 months of age in Example 3 of the present disclosure;

FIG. 10 shows the immunofluorescence co-staining results of different mouse models at different ages in Example 3 of the present disclosure;

FIG. 11 shows the results of gait test, open field test, and rotarod test of each mouse model at 1 month of age in Example 4 of the present disclosure;

FIG. 12 shows the results of gait test, open field test, and rotarod test of each mouse model at 3 months of age in Example 4 of the present disclosure;

FIG. 13 shows the results of gait test, open field test, and rotarod test of each mouse model at 6 months of age in Example 4 of the present disclosure.

### DETAILED DESCRIPTION OF THE EMBODIMENTS

Unless otherwise defined, all technical and scientific terms used herein have the same meaning as commonly understood by those skilled in the technical field of the present disclosure. The terms “comprising” and any variations thereof in the description and claims of the present disclosure are intended to cover non-exclusive inclusion.

The present disclosure addresses the shortcomings of PD animal models in the prior art, especially the problem that PD-like symptoms appear late in the Thy1-SNCA model. Based on the new discovery that Clu can mediate α-syn degradation, the present disclosure proposes the idea of accelerating the occurrence of PD-like symptoms of the SNCA gene by knocking out Clu, and then successfully constructs the Thy1-SNCA;Clu−/− mouse model. The construction method includes the following steps:
(1) constructing a Clu gene knockout mouse model, referred to as the Clu-KO mouse;
(2) subjecting a Thy1-SNCA mouse to crossing with the Clu-KO mouse to obtain an F1 generation, and then conducting identification to obtain a Thy1-SNCA;Clu−/+ mouse; and
(3) subjecting the Thy1-SNCA;Clu−/+ mouse in F1 generation to backcrossing with the Clu-KO mouse, and then conducting identification to obtain a mouse with a target genotype (Thy1-SNCA;Clu−/− mouse).

In the construction method, the identification can be completed by using rat tail alkali lysis.

In the construction method, the Thy1-SNCA mouse is an existing PD model, which can be purchased directly through commercial channels or constructed by oneself according to existing technologies, such that the construction method will not be described in detail here.

The technical solution in the present disclosure will be clearly and completely described below in conjunction with the examples of the present disclosure. It should be understood that the specific embodiments described herein are merely intended to illustrate and explain the present disclosure, rather than to limit the present disclosure.

If specific techniques or conditions are not indicated in the examples, the procedures shall be conducted in accordance with the techniques or conditions described in the literature in the art or in accordance with the product specification. Reagents or instruments not specified with manufacturers are all conventional products that can be purchased through formal channels.

### Example 1 Construction of Clu-KO Mice

The Clu gene (gene ID: 12759) was located on chromosome 14 (14D1) and had five transcripts. A fifth transcript (NP_038520.2) contained a complete conserved consensus coding sequence (CCDS). The transcript encoded 9 exons, with a translation start site ATG located in exon 1 and a translation stop site TGA located in exon 9. The transcript was 1,645 bp and encoded 448 amino acids.

In the present disclosure, the fifth transcript design was selected based on CRISPR/Cas9 gene editing to knock out the E3 to E4 regions of exon (with a total length 2,636 bp, the specific base sequence shown in SEQ ID NO: 1), thereby constructing the Clu-KO mice.

The present disclosure included the following steps:

(1) gRNAs were designed, constructed, and in vitro transcribed according to the design plan.

Target sequences of gDNAs were:
gRNA-B1 (matching the gene strand sequence):

(SEQ ID NO: 2)
ATGTGTCTGGCAACCGCATCAGG;

gRNA-B2 (matching the gene strand sequence):

(SEQ ID NO: 3)
CCCTACCTGTGAGATTCACTTGG.

(2) A homologous recombination vector (namely Donor vector) was constructed.

According to step (1), the 2 synthesized single-stranded oligonucleotide gRNA sequences were annealed to form a double-stranded DNA to construct a gRNA expression vector. The recombinant plasmid was transformed into DH5a competent cells, and a positive clone plasmid was screened and identified by kanamycin resistance and target DNA sequencing. The correct colony clone was selected, and a plasmid was extracted after expansion culture to prepare an in vitro transcription template.

A recombinant plasmid (Donor vector) carrying the target site homology region and the target fragment was prepared and transformed into DH5a competent cells. A positive clone plasmid was screened and identified by ampicillin resistance and sequencing of the inserted fragment. The correct colony clone was selected, a plasmid was extracted and purified after expansion culture, and the obtained donor fragment product was used for injection.

(3) Clu-KO mice were obtained.

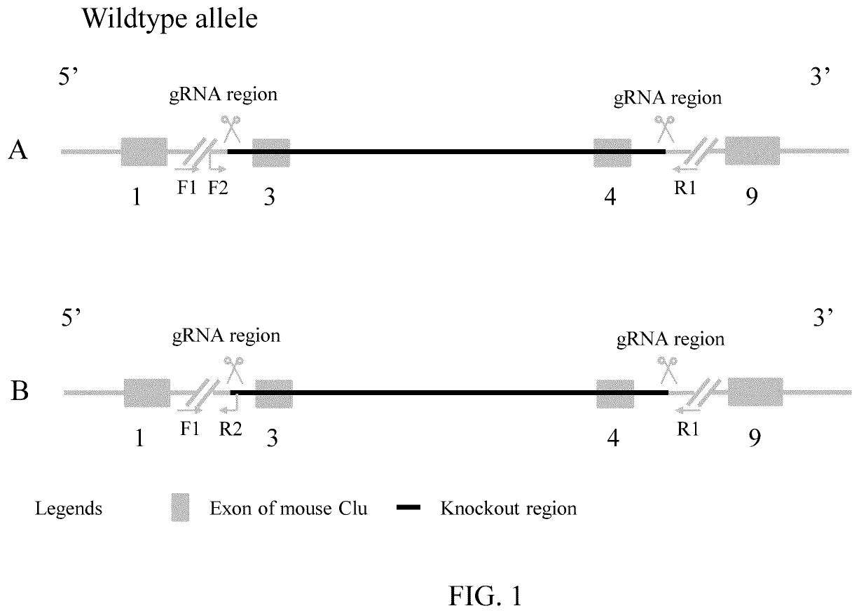

The CRISPR/Cas9 system and the Donor vector sample were microinjected into the fertilized eggs of female C57BL/6N background mice, and the fertilized eggs were transplanted into pseudo-pregnant female mice to obtain recipient mice, which were then allowed to become pregnant and give birth. The F0 generation pups born by the recipient mice were numbered after their tails and toes were cut at 5-7 d, and their genomic DNA was extracted for PCR and sequencing to confirm their genotypes. A genotype identification strategy of the Clu-KO mice was shown in FIG. 1, and the PCR primers used included the following two pairs of primers:
PCR primer 1 (annealing temperature 60° C.):

F1:
(SEQ ID NO: 4)
5′-AACCTCTTGCTCATAGCTGACC-3′,
R1:
(SEQ ID NO: 5)
5′-GACCCAAGTTCCCACTGTTCTG-3′;

PCR primer 2 (annealing temperature 60° C.):

F1:
(SEQ ID NO: 4)
5′-AACCTCTTGCTCATAGCTGACC-3′,
R2:
(SEQ ID NO: 6)
5′-AGTCACAGCTCAGAAACTCAGG-3′.

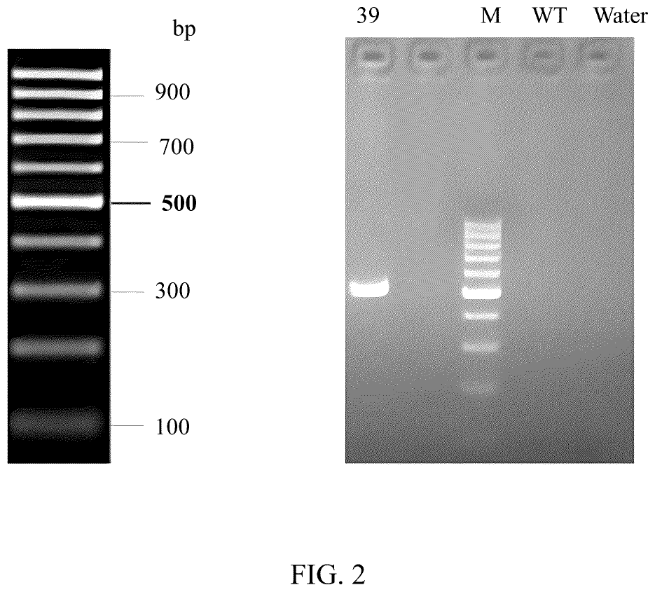

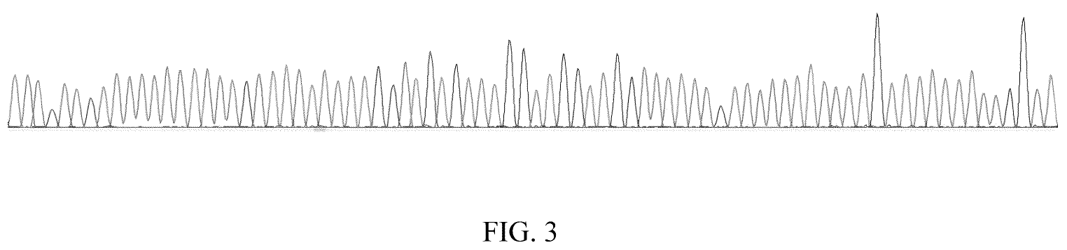

When the PCR primer 1 was used for detection, a product length of 514 bp (FIG. 2) indicated that the target region had been knocked out (the length of the product without knockout was 3,150 bp). The positive animal (the mouse No. 39 in FIG. 2) was further sequenced using a sequencing primer (F2 in FIG. 1A, with a sequence of GTTCTCTCAGCACACTTCAAGG (SEQ ID NO: 11)). The sequencing results were shown in FIG. 3, indicating that the 2,636 bp fragment shown in SEQ ID NO: 1 had been deleted.

### Example 2 Construction of Thy1-SNCA;Clu−/− Mice

The Clu-KO mice prepared in Example 1 were used to prepare Thy1-SNCA;Clu−/− mice, including the following steps:

(1) Thy1-SNCA;Clu−/+ mice were obtained by crossing.

After F0 mice with positive Thy1-SNCA reached sexual maturity, they were mated with Clu−/− background mice (the Clu-KO mice). The F1 mice were separated into cages on day 21, and their tails and toes were cut to allow numbering. Genomic DNA was extracted and PCR was conducted using the primers shown in Table 1 (the reaction system and reaction procedures were shown in Tables 2 and 3) to confirm the genotype.

TABLE 1
Information of primers used for Thy1-
SNCA genotype identification

| Primer | | Product |
|---|---|---|
| name | Primer sequence (5′-3′) | size |
| SNCA-F1 | CTTTCTCTGAGTGGCAAAGGA | About |
| | (SEQ ID NO: 7) | |
| | CCACACCCTGTTTGGTTTTC | 250 bp |
| | (SEQ ID NO: 8) | |
| SNCA-F1 | GCATGATCCTGAGACGGACT | 329 bp |
| | (SEQ ID NO: 9) | |
| | TGCTCTGGGCTCCATTTC | |
| | (SEQ ID NO: 10) | |

TABLE 2
PCR reaction system used for Thy1-SNCA genotype identification

| Component | Volume (μL) |
|---|---|
| 2× Tap Master Mix | 12.5 |
| ddH₂O | 4.5 |
| Forward primer (10 pmol/μL) | 1 |
| Reverse primer (10 pmol/μL) | 1 |
| Genomic DNA of mouse tail | 1 |
| Total volume | 20 |

TABLE 3
PCR reaction procedure used for
Thy1-SNCA genotype identification

| Procedure | Temperature | Time | | Number of cycles |
|---|---|---|---|---|
| 1 | 95° C. | 5 | min | 1 |
| | 95° C. | 30 | s | |
| 2 | 62° C. | 30 | s | 35 times |
| | 72° C. | 30 | s | |
| 3 | 72° C. | 10 | min | 1 |
| 4 | 4° C. | 1 | h | 1 |

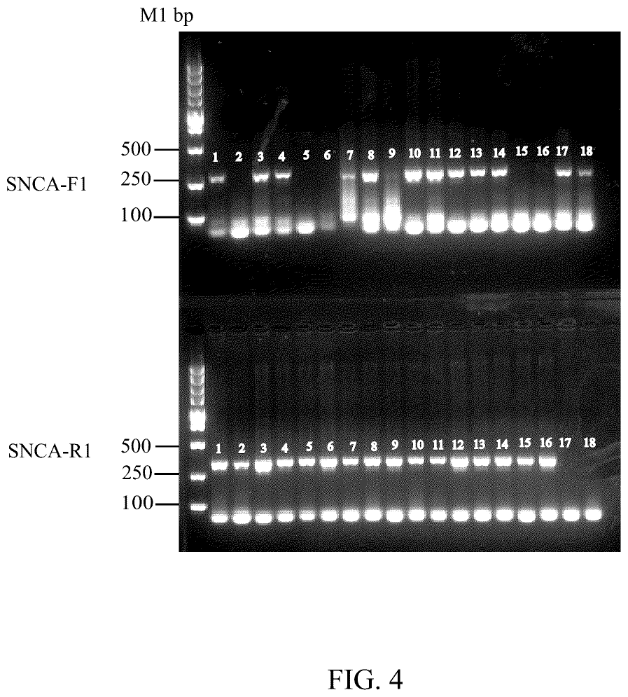

Some of the identification results of F1 generation mice were shown in FIG. 4. As shown in FIG. 4, mice numbered 3, 4, 8, 10, 11, 12, 13, 14, and 17 were all positive (namely Thy1-SNCA;Clu−/+).

(2) Thy1-SNCA;Clu−/− mice was constructed.

Thy1-SNCA;Clu−/+ adult mice were selected to allow backcrossing with Clu−/− mice to obtain F2 generation mice, and Thy1-SNCA genotype and Clu-KO genotype of the F2 generation mice were identified to obtain Thy1-SNCA;Clu−/− mice.

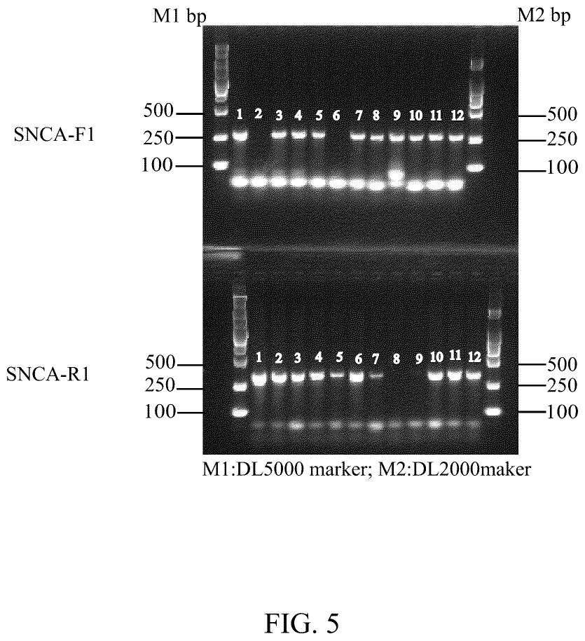

The identification of Thy1-SNCA genotype was the same as that in step (1). Part of the electrophoresis detection results of F2 generation Thy1-SNCA mice were shown in FIG. 5: mice numbered 1, 3, 4, 5, 10, 11, and 12 were Thy1-SNCA positive, and the remaining numbered mice were wild-type (WT).

The Clu-KO genotype identification strategy was shown in FIG. 1B and the primers used were shown in Table 4. In Table 4, primer AB-Clu was F1 in FIG. 1B, A-Clu was R1 in FIG. 1B, and B-Clu was R2 in FIG. 1B. The PCR reaction system and reaction procedure used for Clu-KO genotype identification were shown in Tables 5 and 6, respectively.

TABLE 4
Information of primers used for Clu-KO
genotype identification

| Primer | | | Product |
|---|---|---|---|
| name | Primer sequence (5′-3′) | Type | length |
| AB-Clu | AACCTCTTGCTCATAGCTGACC | Common | / |
| | (SEQ ID NO: 4) | | |
| A-Clu | GACCCAAGTTCCCACTGTTCTG | Reverse | 514 bp |
| | (SEQ ID NO: 5) | | |
| B-Clu | AGTCACAGCTCAGAAACTCAGG | Reverse | 749 bp |
| | (SEQ ID NO: 6) | | |

TABLE 5
PCR reaction system used for Clu-KO genotype identification

| Component | Volume (μL) |
|---|---|
| 2× Tap Master Mix | 12.5 |
| ddH₂O | 4.5 |
| Forward primer (10 pmol/μL) | 1 |
| Reverse primer (10 pmol/μL) | 1 |
| Genomic DNA of mouse tail | 1 |
| Total volume | 20 |

TABLE 6
PCR reaction procedure used for Clu-KO genotype identification

| Procedure | Temperature | Time | | Number of cycles |
|---|---|---|---|---|
| 1 | 95° C. | 5 | min | 1 |
| | 95° C. | 30 | s | |
| 2 | 62° C. | 30 | s | 35 times |
| | 72° C. | 30 | s | |
| 3 | 72° C. | 10 | min | 1 |
| 4 | 4° C. | 1 | h | 1 |

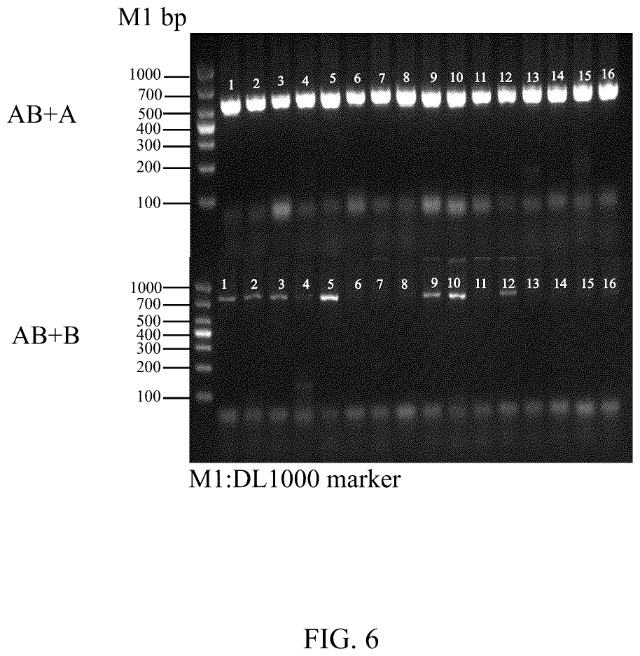

When the two pairs of primers shown in Table 4 were used for detection, the homozygote had only one band, namely 514 bp, while the heterozygote had two bands, namely 514 bp and 749 bp. Specifically, some of electrophoresis detection results of F2 generation Clu mice in this example were shown in FIG. 6: mice numbered 4, 6, 7, 8, 11, 13, 14, 15, and 16 were Clu−/−, while mice numbered 1, 2, 3, 5, 9, 10, and 12 were Clu−/+.

### Example 3

In this example, pathological examination of Thy1-SNCA mice, Thy1-SNCA;Clu−/+ mice, and Thy1-SNCA;Clu−/− mice was conducted, including the following steps:

### (1) Preparation of Brain Tissue Sections

The mice were perfused transcardially with 4% paraformaldehyde (PFA) in 0.1 M phosphate-buffered saline (PBS, pH=7.4) under anesthesia with isoflurane and chloral hydrate (3%). Harvested brain tissues were fixated in a same fixative overnight at 4° C., dehydrated with 30% sucrose in PBS, and serially sectioned on an oscillating microtome (Leica).

(2) Detection of Phosphorylated α-Syn (p-α-Syn) Aggregation Using Thioflavin (Ths) Staining

To detect the co-localization of p-S129α-syn and ThS in the mouse brain, the sections were incubated with mouse anti-p-S129α-syn primary antibody (Abcam, #ab51253, 1:500) at 4° C. overnight and washed with PBS 3 times, 5 min each time. The sections were incubated with Alexa Fluor 594-labeled anti-mouse antibody at room temperature for 1 h. The sections were rinsed briefly in PBS, stained with 0.5% ThS in 50% ethanol for 5 min, then washed with 50% ethanol and placed in distilled water. The sections were mounted with DAPI and the slides were covered with cover glass, and examined under a confocal microscope (Leica Microsystems STELLARIS 5, Germany). The experiment was repeated at least three times.

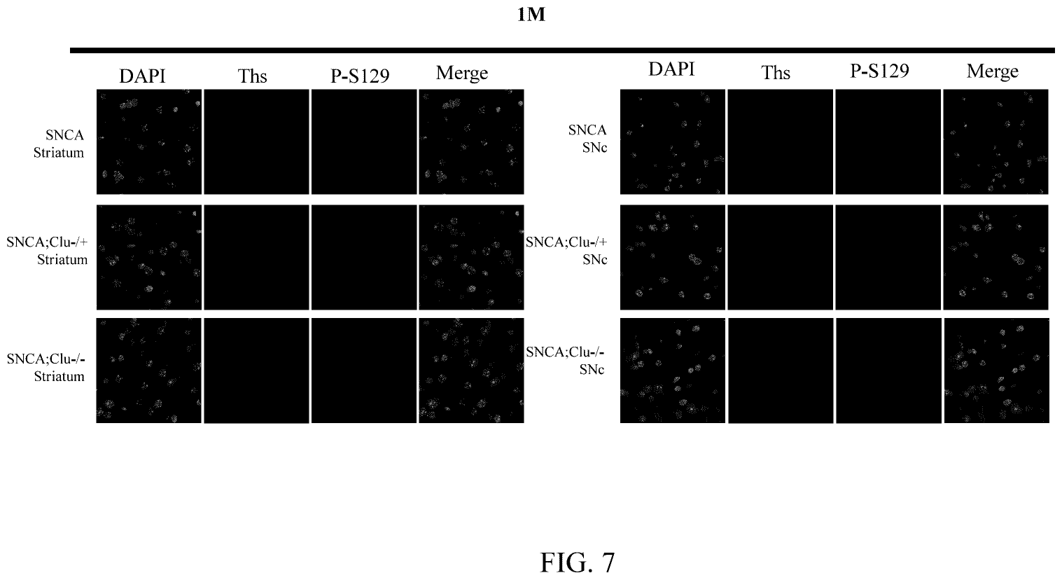

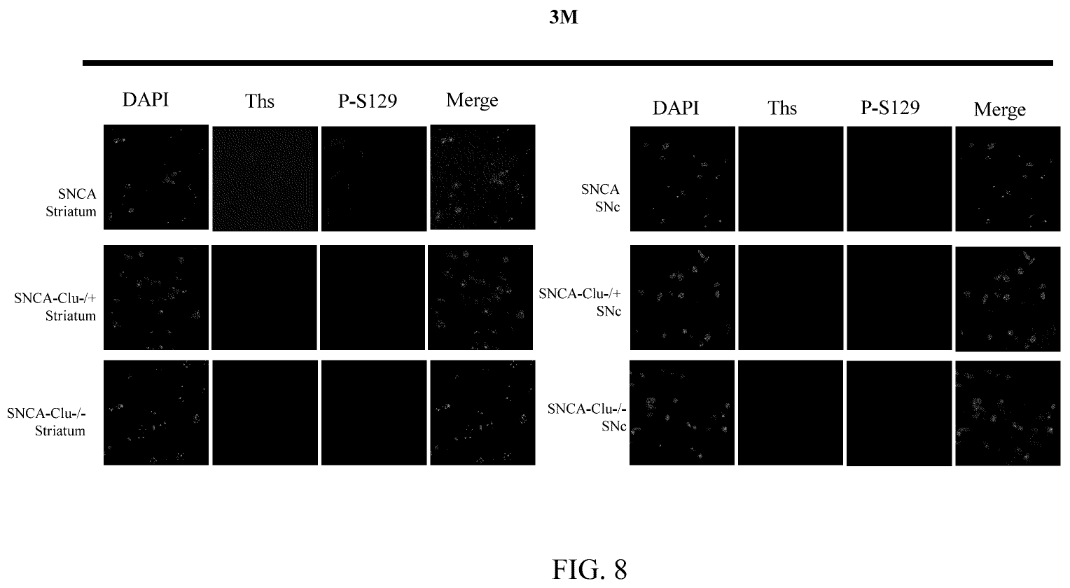

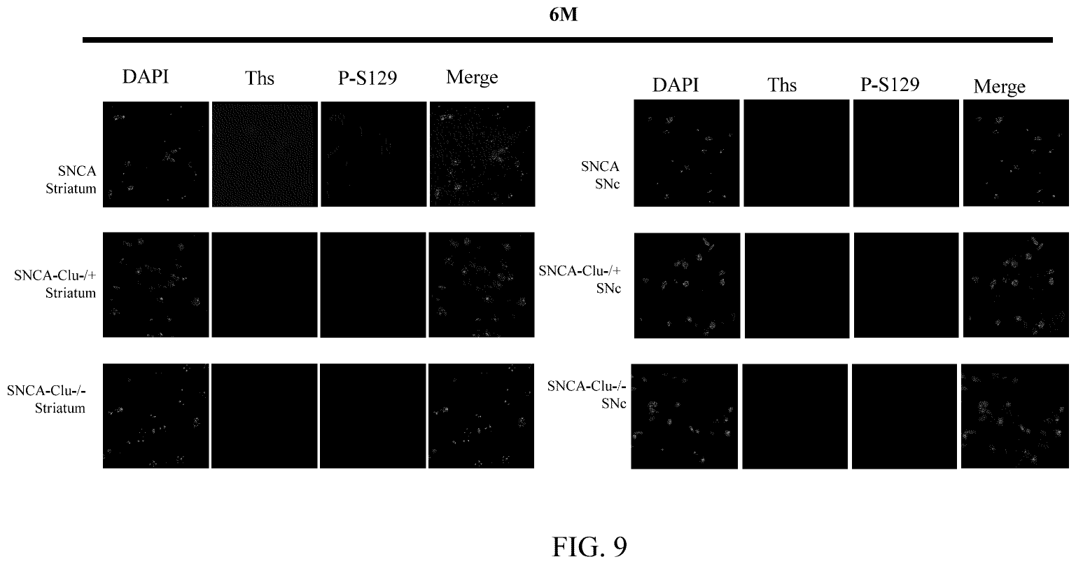

The experimental results were shown in FIG. 7 to FIG. 9: At 1 month of age (FIG. 7) and 3 months of age (FIG. 8), there was no significant difference in the p-α-syn aggregated in the three genotypes of Thy1-SNCA, Thy1-SNCA;Clu−/+, and Thy1-SNCA;Clu−/− mice. At 6 months of age (FIG. 9), a large amount of p-α-syn aggregated appeared in the substantia nigra and striatum of Thy1-SNCA;Clu−/− mice. This indicated that compared with Thy1-SNCA (14 M), the Thy1-SNCA;Clu−/− mice provided in the present disclosure could show obvious PD pathology at an early stage (6 M).

(3) Co-Staining of TH and p-α-Syn by Immunofluorescence

The sections were permeabilized in PBS/0.1% Triton X-100 for 10 min and blocked with 4% normal bovine serum in PBS/0.1% TritonX-100 for 1 h at room temperature. The sections were incubated with primary antibodies p-S129 (Abcam, #ab51253, 1:500) and TH (CST #58844S) in 2% serum in PBS/0.1% Triton X-100 at 4° C. overnight. The sections were washed with PBS and incubated with Alexa Fluor 594- and Alexa Fluor 488-conjugated secondary antibodies (namely TH secondary antibody and P-α-syn secondary antibody, Invitrogen) for 1 h at room temperature. After washing with PBS, the sections were incubated with DAPI to stain cell nuclei. Images were captured using a Zeiss upright microscope.

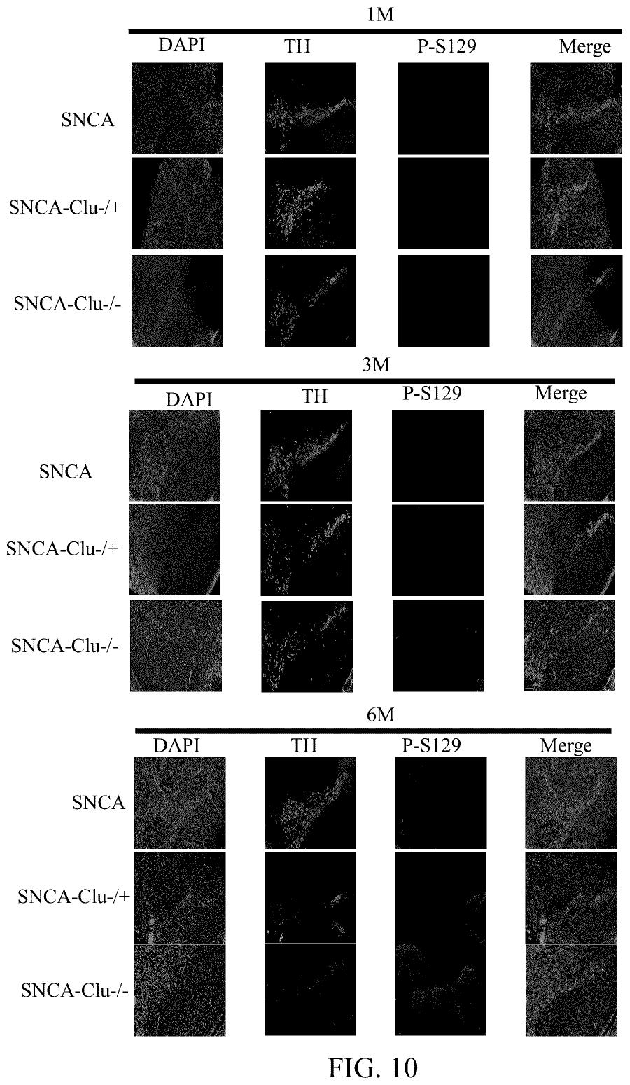

The results were shown in FIG. 10: with the increase of age, in 6 M Thy1-SNCA;Clu−/− mice, a large amount of TH was lost and p-α-syn showed obvious aggregation.

The above results indicated that the Thy1-SNCA;Clu−/− model mice could well replace Thy1-SNCA mice; and compared with Thy1-SNCA mice, the onset of PD-like symptoms was advanced to 6 months of age, greatly shortening the experimental period.

### Example 4

The motor abilities of PD patients are generally impaired, always manifested as resting tremor, changes in balance and gait. Therefore, this example examined the behavior of Thy1-SNCA mice, Thy1-SNCA;Clu−/+ mice, and Thy1-SNCA;Clu−/− mice at different ages.

Open field test: in the open field test, mice were placed in a 40×40 square grid and allowed to move freely for 10 min. Their activity trajectory during the period, as well as the number and time of entering the central area, were recorded.

Rotarod test: in the rotarod test, mice were trained on a rotarod apparatus (MED-Associates) at 20 rpm for 5 min. After the initial training, the mice were tested 2 times with the speed increasing from 10 rpm to 20 rpm. The training lasted for 3 d, with the speed increasing from 20 rpm to 30 rpm on the second day and from 30 rpm to 40 rpm on the third day. The time to fall off was recorded on the fourth day of the test.

Gait test: in the gait test, mice were placed into the gait machine from the left side and allowed to freely walk through the passage to the dark box on the right side. Mice were trained for three consecutive days, with three consecutive trials per training session. On the fourth day, the mice were tested and their trajectory of the gaiter was recorded, including step base, stride length, and number of steps.

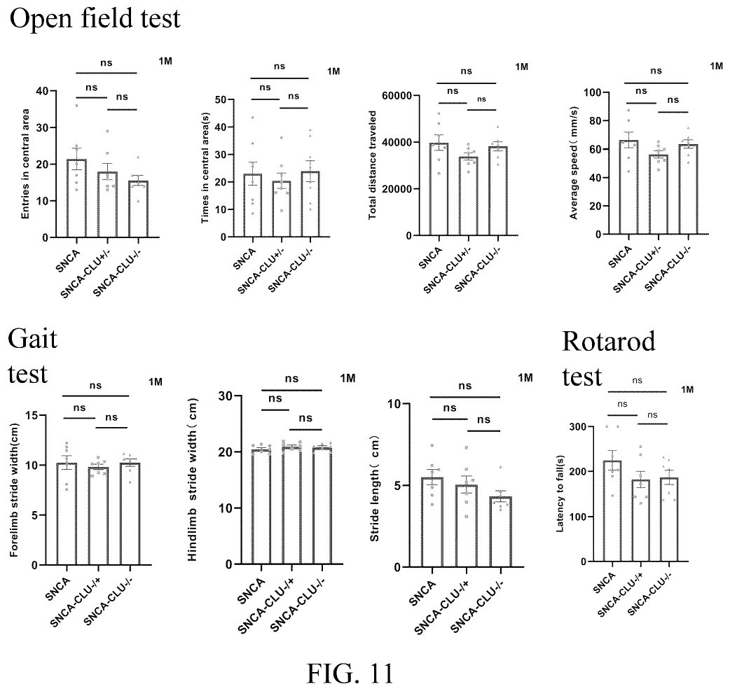

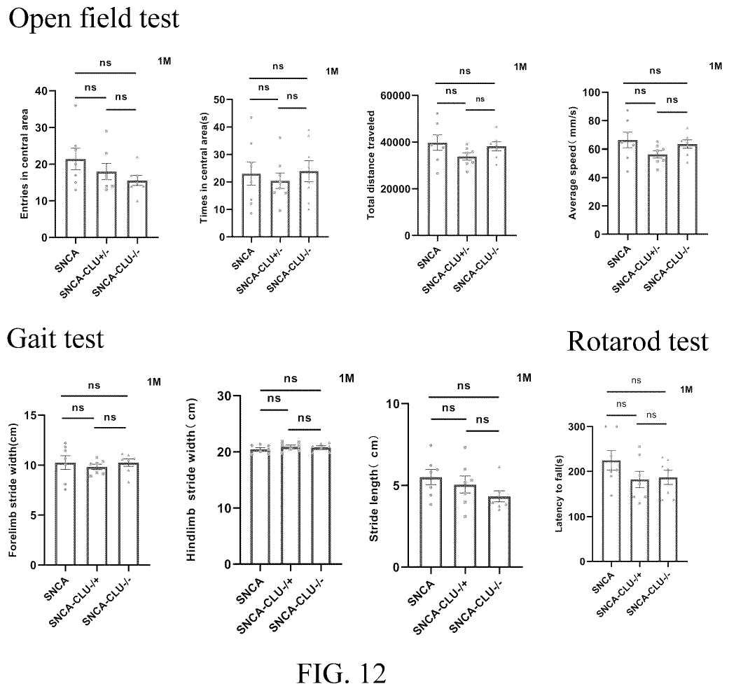

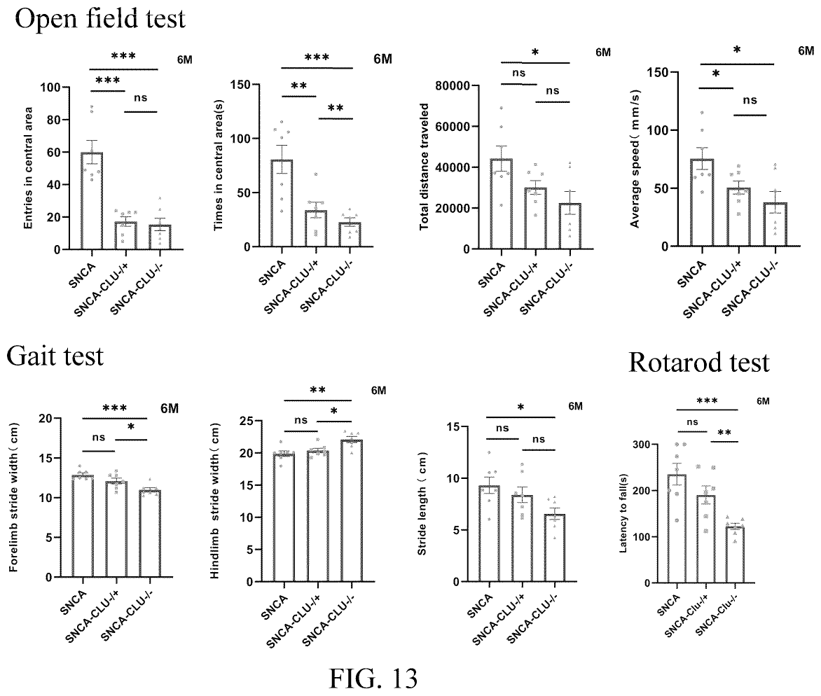

The test results were shown in FIG. 11 to FIG. 13. In the open field test, there was no statistically significant difference between the three groups of animals at 1 month and 3 months of age. However, there was a difference in the number of times the 3-month-old Thy1-SNCA;Clu−/− crossed the central platform, and the difference was more significant at 6 months of age. In the rotarod test, there was no difference in the grasping ability of Thy1-SNCA;Clu−/− mice at 1 month and 3 months of age, but there was a significant obstacle at 6 months of age. Compared with 6 M Thy1-SNCA mice, Thy1-SNCA;Clu−/− mice were significantly smaller in step base and stride length than Thy1-SNCA mice, and the number of steps increased significantly. This was because PD patients showed “small steps”-like signs (shortened stride length, increased number of steps) when they had movement disorders.

The above data showed that there was no difference between the three groups of mice at an earlier age (1-5 M). However, SNCA-Clu-KO mice showed significant motor impairment at 6 months of age, nearly 8 months earlier than the onset of motor impairment in Thy1-SNCA mice.

In summary, the Thy1-SNCA;Clu−/− mice constructed in the present disclosure can show obvious PD pathology at 6 months of age compared with the traditional PD model, providing more model mice for PD research.

It should be pointed out that the described examples are merely a part rather than all of the examples of the present disclosure. All other examples obtained by a person of ordinary skill in the art based on the examples of the present disclosure without creative efforts should fall within the protection scope of the present disclosure.

What is claimed is:

1. A construction method of a Thy1-SNCA;Clu gene knockout mouse model, comprising the following steps:
subjecting a Thy1-SNCA mouse to crossing with a Clu-KO mouse to obtain an F1 generation, and then conducting identification to obtain a Thy1-SNCA;Clu−/+ mouse; and
subjecting the Thy1-SNCA;Clu−/+ mouse to backcrossing with the Clu-KO mouse to obtain an F2 generation, and then conducting identification to obtain a mouse with a target genotype, recorded as a Thy1-SNCA;Clu−/− mouse.

2. The construction method according to claim 1, wherein the Clu-KO mouse lacks a sequence shown in SEQ ID NO: 1 relative to a wild-type mouse.

3. The construction method according to claim 2, wherein the Clu-KO mouse is obtained by conducting gene knockout on the sequence shown in SEQ ID NO: 1 in a Clu gene of the wild-type mouse using a CRISPR/Cas9 system.

4. The construction method according to claim 3, wherein the gene knockout comprises: microinjecting the CRISPR/Cas9 system and a homologous recombination vector into a fertilized egg of a female C57BL/6N background mouse, transplanting the fertilized egg into a pseudo-pregnant female mouse to obtain a recipient mouse, allowing the recipient mouse to become pregnant and give birth to a newborn mouse of a F0 generation, and then extracting a genomic DNA of the newborn mouse to allow identification to obtain the Clu-KO mouse.

5. The construction method according to claim 4, wherein gRNA target sequences in the CRISPR/Cas9 system are shown in SEQ ID NO: 2 and SEQ ID NO: 3.

6. The construction method according to claim 4, wherein primers for identifying the Clu-KO mouse have sequences shown in SEQ ID NO: 4 and SEQ ID NO: 5.

7. The construction method according to claim 1, wherein a primer set for identifying the Thy1-SNCA;Clu−/+ mouse comprises primers having sequences shown in SEQ ID NO: 7 to SEQ ID NO: 10.

8. The construction method according to claim 7, wherein a primer set for identifying the mouse with the target genotype comprises primers having sequences shown in SEQ ID NO: 4 to SEQ ID NO: 10.

9. An application of a Thy1-SNCA;Clu gene knockout mouse model constructed according to the construction method of claim 1 in preparation of a model for Parkinson's disease (PD), comprising the following steps:
S1: gait training the mouse model for 3 days with each mouse crosses a channel for 3 times, and behavioral testing for the mouse model in 4th day to study disease mechanism of PD; and
S2, deeply anaesthetizing and sequentially perfusing the mouse model with saline and 4% paraformaldehyde for immunohistochemistry staining; taking out brain tissues of the mouse model, infiltrated the brain tissues with paraffin and cutting into serial sections; deparaffinizing the sections in xylene, hydrated through descending ethanol; steeping the sections in antigen retrieval solution, and incubating in hydrogenperoxide to eliminate endogenous peroxidase activity; blocking the section in bull serum albumin (BSA) at room temperature; incubating the sections with primary antibodies and then rinsing three times in phosphate buffer saline (PBS); applying antibodies to the sections for immuno-fluorescence staining; staining in 4′,6-diamidino-2-phenylindole (DAPI) solution; and capturing images of the sections using microscope for pathological detection.


SEQUENCE LISTING (11 sequences)

SEQ ID NO: 1 - DNA, 2636 bp, synthetic construct
  source 1..2636: mol_type = other DNA
caaccgcatcaggtagaaaacagcagaggtgtgggaaatggcacttgacacaggataagtaagtggtaccaccgtgacatgatgtgtcattgccttcgtcacctggggccgtgtcatggctatggtctccatggtgaatgcagctcttcatttatcgtcccgtctccatggtgtctggtgctatcctgtcacctgaccctgtgcgttgtagcaaagaagtgccaggattgggttaacctgagcttcacagagataatttccaaaagagcacttttattacaccttgatgcttaggaggttgttttgttttgttttctgttttccttgaaatattctctgtggctgttggctctccagggtggcatggtggggtcagtcatagtatggctgcccctctctgcaccactcactcctgagtttctgagctgtgacttggagtctaccatgagccctttgatgggacatagaggcaggactgaggccataaggactgccacaggcttggggtaatagtgatggtgtctcagcttgacgcccccagcactccccatcaaagcatggctttctcaccgtccccctggcacccaggctatttctccagccattcctccccgcccccctcaactcactgtaccttcctctctctcacagaactgtccactcaagggagtaggtatattaataaggagattcagaacgccgtccagggagtgaagcacataaaaactctcatagaaaaaaccaacgcagagcgcaagtccttgctcaacagtttagaggaagccaagaagaagaaagaggtagggccaagccttggctgagcccaggctctccctgctggaggggaggaggggacatggtgagcaggtgcctttctgggttgccatggtgtcctaaggcggtcctcgaagggttccgctaatgctggactgaggagccagggaaagcacattcgcttgtgggagcctcagtgccgaggccttgaatttggtttccctgcaactctggtccagggcctgaagcctagattcatttcaataggtttttctaacccttgctgtgggtagggcatggtgcaggactcagcggtgtgtgtgtgtgtgtgtgtgtgtgtgtgtgtgtgtgatatgagcatccagacatttcccattcttttcttctgtattcaaaatgacttttaacatgaaagttcttattgaaatattttaaaatcaatgtaaaattcttgtaatctatccttagacatccatccattcattcattcccactcagatataggggaggggggcagttttcaaaacccttagtagaagctgcagttgtggctagcaccaggccctgcagagattgtgcttctgtaatttataaattacagacagtaaggtatgagcagccacaataaaatagagcaattataatgctataccagttataccatacagaaggctacgcagatgtgatttctcccacacaaaatatctcaattcctcagcttccacaatactatttttagactggttgaccatggatagctaagaactgataacaagggactgccctgttaccatttgtttaatttgtcatctacatttgaacatcaatgttgactctggtacacacacacacacacacacacacacacacacacacacacacacacaaacttatccacagattcatccatccacttaattattttccatattgatatgtagacacacacatttgctataaactagtatctcttcaggcggagaatttaaatttttcattttagcaatccccagagccaatgactgggtcaggggcatggtaatgagtttgatggttgatttgactggatagcttcgatttgcatgtcagaggccacagttctaacagaaagataagactgtcctctttggggagatgagaggagacagtggggtggtttaaaggagaggtggggcctaaggaagagacggtcagagcagcagatcagagtggacagggaagcttagggaatggctgggccttgaagaaggagaggctcctgagagcgtggctggggaggcccaacagaagggctagagcgaagctgtgggcaccaggagcaaccttgtactcagatcggggagaccggctgggtctctgtgtgggtcacctggccttcttgtcatctccctgaaggatgctctggaggacactagggattctgaaatgaagctgaaggcattcccggaagtgtgtaacgagaccatgatggccctctgggaggagtgcaagccctgcctgaagcatacctgcatgaagttctatgcacgtgtctgcaggagcggctcagggctggtcggccagcaggtgagaaggagagatgtccacagcctgggttctggatggggaatgtggagaatccatgaaataggcctcattccatatgctgggtacagttgattagcattgcttgactgcacatcgttctagtgtcatggtcgattggtgatgtctgcttggcaggtaccatctctttcaagacctttaactggttgggtaaaatggccttatgggatggtagaatcctgtgttagaataggaaaagggaattaaaaaatatttagtctatttgcattaattctatccctacctgtgagattcac

SEQ ID NO: 2 - DNA, 23 bp, synthetic construct
  source 1..23: mol_type = other DNA
atgtgtctggcaaccgcatcagg

SEQ ID NO: 3 - DNA, 23 bp, synthetic construct
  source 1..23: mol_type = other DNA
ccctacctgtgagattcacttgg

SEQ ID NO: 4 - DNA, 22 bp, synthetic construct
  source 1..22: mol_type = other DNA
aacctcttgctcatagctgacc

SEQ ID NO: 5 - DNA, 22 bp, synthetic construct
  source 1..22: mol_type = other DNA
gacccaagttcccactgttctg

SEQ ID NO: 6 - DNA, 22 bp, synthetic construct
  source 1..22: mol_type = other DNA
agtcacagctcagaaactcagg

SEQ ID NO: 7 - DNA, 21 bp, synthetic construct
  source 1..21: mol_type = other DNA
ctttctctgagtggcaaagga

SEQ ID NO: 8 - DNA, 20 bp, synthetic construct
  source 1..20: mol_type = other DNA
ccacaccctgtttggttttc

SEQ ID NO: 9 - DNA, 20 bp, synthetic construct
  source 1..20: mol_type = other DNA
gcatgatcctgagacggact

SEQ ID NO: 10 - DNA, 18 bp, synthetic construct
  source 1..18: mol_type = other DNA
tgctctgggctccatttc

SEQ ID NO: 11 - DNA, 22 bp, synthetic construct
  source 1..22: mol_type = other DNA
gttctctcagcacacttcaagg